(19) United States
(12) Patent Application Publication
(10) Pub. No.: US 2014/0069171 A1
(43) Pub. Date: Mar. 13, 2014

(54) PORTABLE ENVIRONMENTAL AUDIT TOOL

(71) Applicant: Purkay Laboratories, Inc., North Grafton, MA (US)

(72) Inventor: Indrajit Purkayastha, North Grafton, MA (US)

(73) Assignee: Purkay Laboratories, Inc., North Grafton, MA (US)

(21) Appl. No.: 14/021,535

(22) Filed: Sep. 9, 2013

Related U.S. Application Data
(60) Provisional application No. 61/699,542, filed on Sep. 11, 2012.
(60) Provisional application No. 61/708,924, filed on Oct. 2, 2012.

(51) Int. Cl. G01N 33/00

(52) U.S. Cl. CPC G01N 33/0009
USPC 73/31.01

(57) ABSTRACT

A method and instrument is provided that includes a flexible stick that is adjustable for different heights. The method and instrument includes one or more sensor modules coupled to the flexible stick. The method and instrument includes one or more control systems that establish different thresholds for the one or more sensor modules at different heights. The one or more control systems monitors a plurality of environmental parameters in a predetermined sequence and indicates whether at least one environmental parameter is below, within, or above the thresholds of a plurality of set-points through a display indicator.

### PRIORITY INFORMATION

This application claims priority from provisional application Ser. No. 61/699,542 filed Sep. 11, 2012, and provisional application Ser. No. 61/708,924 filed Oct. 2, 2012, each of which is incorporated herein by reference in its entirety.

### TECHNICAL FIELD

The present disclosure relates to portable environmental instruments for environmental audit purposes.

### BACKGROUND OF THE INVENTION

Several single purpose monitoring devices exist today for environmental monitoring today. Some of these systems are integrated within equipment racks or integrated with the building infrastructure. There are a cadre of devices for portable environment monitoring and data logging purposes, all usually located within a piece of equipment with underlying infrastructure for remote monitoring.

In certain applications, the personnel operating the facility need to have the environmental information at multiple heights of the air around the equipment. This is necessary because air flow is dynamic and there is significant variance in the values. To measure this satisfactorily, today, several measurements need to be made. The existing monitoring systems often do not have sensors present in all areas of the facility. This measurement is often difficult to execute and time consuming, and also suffers from the effect of being conducted at different times; thus, losing the fidelity and accuracy. Some attempts have been made to solve this problem with dedicated wireless sensors to perform the measurement. This is expensive and requires an underlying infrastructure to be installed to read the sensors.

The personnel operating the facilities are interested in solving this problem in a simple inexpensive manner without requiring a significant investment in the facility infrastructure. Furthermore, the need to do so varies from one part of the facility to another. There exists a need for a portable system to perform the measurement in a completely self-sufficient manner without relying on the facility infrastructure.

### SUMMARY OF THE INVENTION

According to one aspect of the invention, there is provided a portable environmental measurement instrument that includes a flexible stick that is adjustable for different heights. The instrument includes one or more sensor modules coupled to the flexible stick. The instrument includes one or more control systems that establish different thresholds for the one or more sensor modules at different heights. The one or more control systems monitors a plurality of environmental parameters in a predetermined sequence and indicates whether at least one environmental parameter is below, within, or above the thresholds of a plurality of set-points through a display indicator.

According to one aspect of the invention, there is provided a method of obtaining and communicating environmental parameters including adjusting a flexible stick that is adjustable for different heights. The method includes coupling one or more sensor modules to the flexible stick. The method includes establishing different thresholds by one or more control systems for the one or more sensor modules at different heights. The one or more control systems monitors a plurality of environmental parameters in a predetermined sequence and indicates whether at least one environmental parameter is below, within, or above the thresholds of a plurality of set-points through a display indicator.

### BRIEF DESCRIPTION OF THE DRAWINGS

FIG. 1 is a perspective view of a portable environmental measurement instrument located near an industrial data center;

FIG. 2 is a schematic diagram illustrating a process and need for accurate monitoring of environmental air quality at different heights;

FIGS. 3A-3B are perspective views of the portable environmental measurement instrument in a first implementation;

FIG. 4 is a perspective view of the portable environmental measurement instrument in another implementation;

FIG. 5 is a schematic diagram of a sensor module illustrating a user interface;

FIG. 6 is a schematic diagram of a sensor module illustrating an on/off switch;

FIG. 7 is a schematic diagram of a sensor module illustrating a plurality of ports;

FIG. 8 is a schematic diagram illustrating remote alert transmitter and receiver modules;

FIG. 9 is a schematic diagram of a sensor module illustrating a battery cover and batteries; and

FIG. 10 is a schematic diagram illustrating an implementation of the invention with a remote web service operating in a cloud environment.

### DETAILED DESCRIPTION OF THE INVENTION

The invention provides a portable environmental monitoring system, to be used by, for example, a facility engineer or the like. It allows monitoring of different environmental parameters with a simple system that is portable. In addition, the invention provides the ability to measure at multiple heights. The system provides the user (or operator) with the ability to determine whether an environmental parameter is within limits or just barely or significantly over the limits. An implementation of the invention consists of a flexible stick which is adjustable; this allows sensors to be at different heights for the user. The user may set high and low limits for each of the specific sensors. The user may set the sampling frequency and the duration. The environmental instrument (or instrument) is then set to monitor the environment. At the end of the interval, the instrument indicates through a flashing LED means or display indicator whether the sensed environmental parameter or parameters were within, slightly or significantly over prescribed limits.

Facility engineers (or the like) are extremely busy personnel. It is expected that they will examine the system from afar simply by looking at the state of the flashing or display indicator, for example, during their walk-through of the facility. Unless the instrument visually indicates there is a reason for concern, the engineer will consider the area under control and may choose to move the instrument elsewhere in the system.

The instrument allows independent monitoring at a plurality of heights, for example, at three different heights. Such environmental parameters include temperature, humidity, noise, air quality, dust, CO₂, CO, radon, ozone, air pressure, air flow, and water. The monitoring at different heights may or may not be of the same environmental parameters. The user is able to set the control limits for each environmental parameter separately. The instrument will monitor the environmental parameter for a defined interval, which may be hours or days depending on the situation. The engineer may be interested in a quick go/no-go assessment without setting up an infrastructure to monitor, or post-processing fair amount of data. This instrument allows the user to quickly assess whether the specific environment is under control or not and to make a determination for the three different heights at the same time, and independently alert the user of the issues present at each height separately.

According to one implementation of the invention, the method employed involves a single processing unit at a base connected with sensors on the flexible stick. The user programs all three sensors simultaneously from the programming unit at the base of the unit. The sensors are wired to the base through a flexible cable mounted within the unit. The implementation allows for a flexible cable or wire to accommodate the different heights of the sensor locations. The data collected by each sensor is visible at a user interface at the base unit. The data collected may be stored in addition on a user supplied memory disk attached to a port on the base. The stored data may he analyzed separately in a different computer later for analysis purposes.

According to another implementation of the invention, sensors are located in independent modules at three different heights. Each sensor module contains its sensors, processing logic and display mechanism. Each sensor module may be located at different heights depending on the application. The data collected by each sensor may be stored in an external memory disk, one for each module, again for post-processing purposes.

In both implementations, a set of warning lights or display indicators alerts the user as to whether the measured environmental parameters are within limits or require attention by the user. Both implementations possess a radio frequency transmitter capability to allow for the warning condition to be transmitted to a remote receiver to be connected to a building management warning system(s).

One of the issues with environmental measurement is that the sensor may not exactly be located where the air might be. The thermal mass of the sensing instrument creates a barrier for the measurement to be real-time as one may want it to be. This delays the responsiveness of the measurement in responding to rapid changes in the environmental conditions. Both implementations use a miniature fan to draw external air into the sensors; thus, eliminating the latency issue and allowing the instrument to provide real-time measurement as possible.

In both implementations, an Ethernet interface is provided for the remote transmittal of data collected by the instrument for display, and processing by a remote computing device, such as a cloud computing service.

The details of one or more implementations of the disclosure are set forth in the accompanying drawings and the description below. Other aspects, features and advantages will be apparent form the description and drawings, and from the claims.

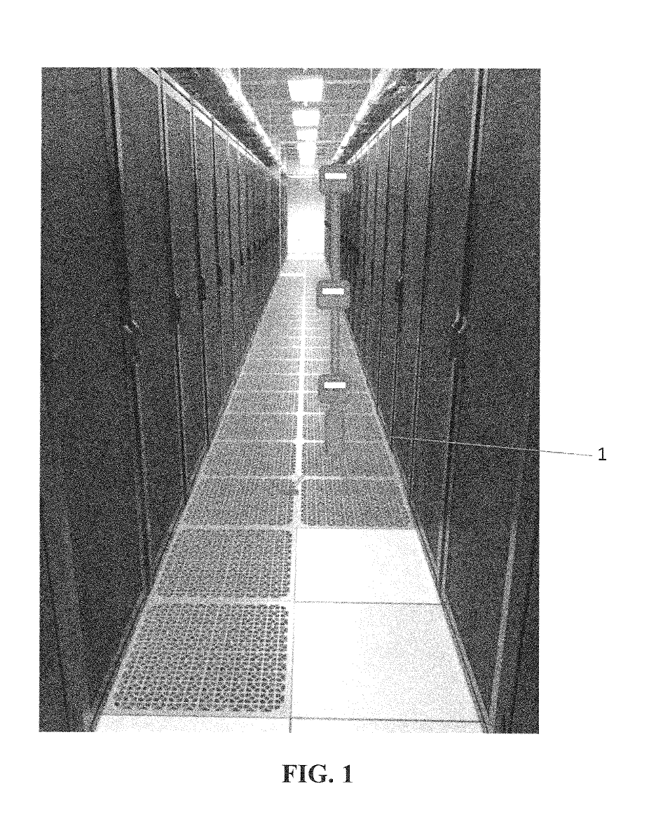

Referring to FIG. 1, a first implementation of a portable environmental measurement instrument 1 is present, for example, in a data center, which may be locations where the environmental parameters such as temperature, humidity, air quality, dust level of the air entering the servers are of critical importance. In certain implementations, cold air is pumped under the floor to rise to the computer servers through grates in the ground. An air conditioner pumps out the air at a certain temperature and humidity. As the air rises through the floor, the values change. The temperature of the air going into the bottom part of the equipment is quite different from the air entering the computer servers in the top of the equipment.

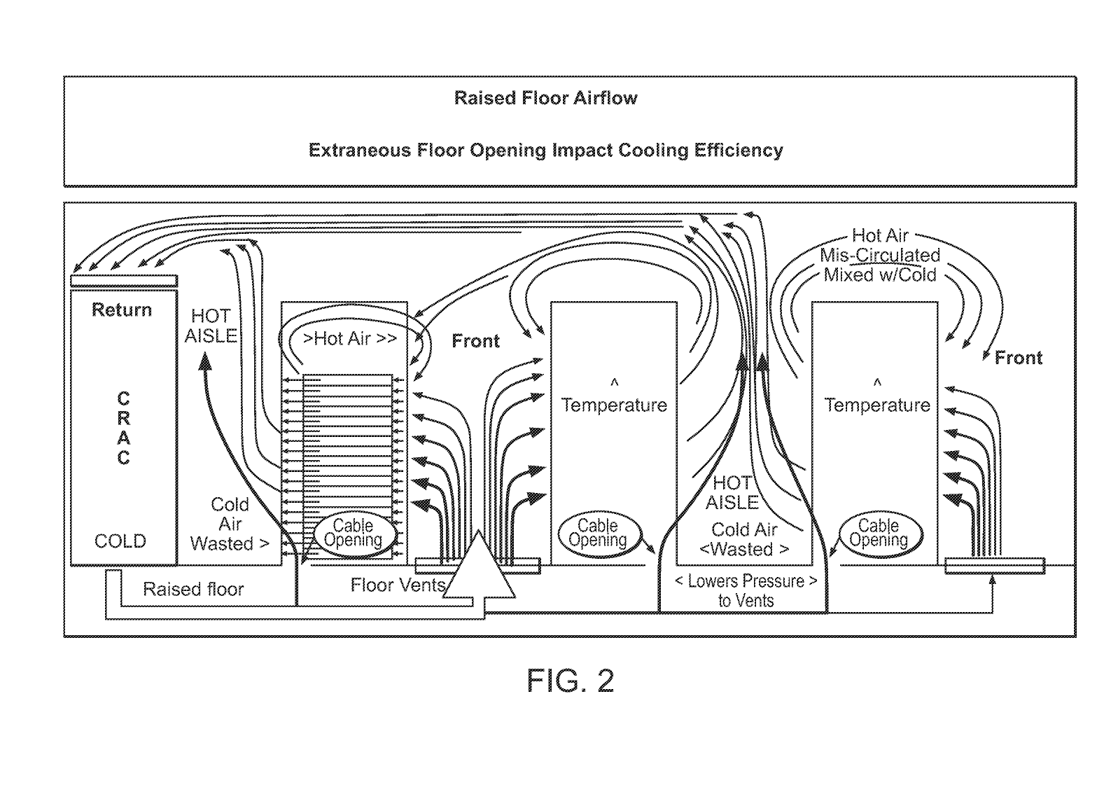

Referring to FIG. 2, there may exist some bleed-through effects of hot air exhausting at the back of the rack entering through the top or the side of the equipment mixing with the cold air; thus, affecting the thermal characteristics of the air going into the server racks. Although one may mount plenty of sensors within the equipment racks, there is no easy way to measure environmental parameters of the air going into the equipment. The only possible means to do so is to place a few tactically-placed wireless sensors. This requires additional back-end infrastructure work to collect the data, detailed knowledge of where the sensors are to be placed, and attention to on-going maintenance issues because these sensors are battery-powered. For this reason, wireless sensors are used only if there are no other choices. Wireless sensors require an accompanying receiver and a monitoring station to read what is being measured by the sensor. Depending on the environment, distance—additional hardware such as repeaters may be needed to let the sensor communicate to the monitoring station without any issues. This requires additional installation within the infrastructure. The invention cites an implementation that addresses this need. The invention provides a means for the user to collect the information without requiring additional infrastructure. The invention is portable; when a monitoring function is performed, the instrument may be moved to a different place of interest.

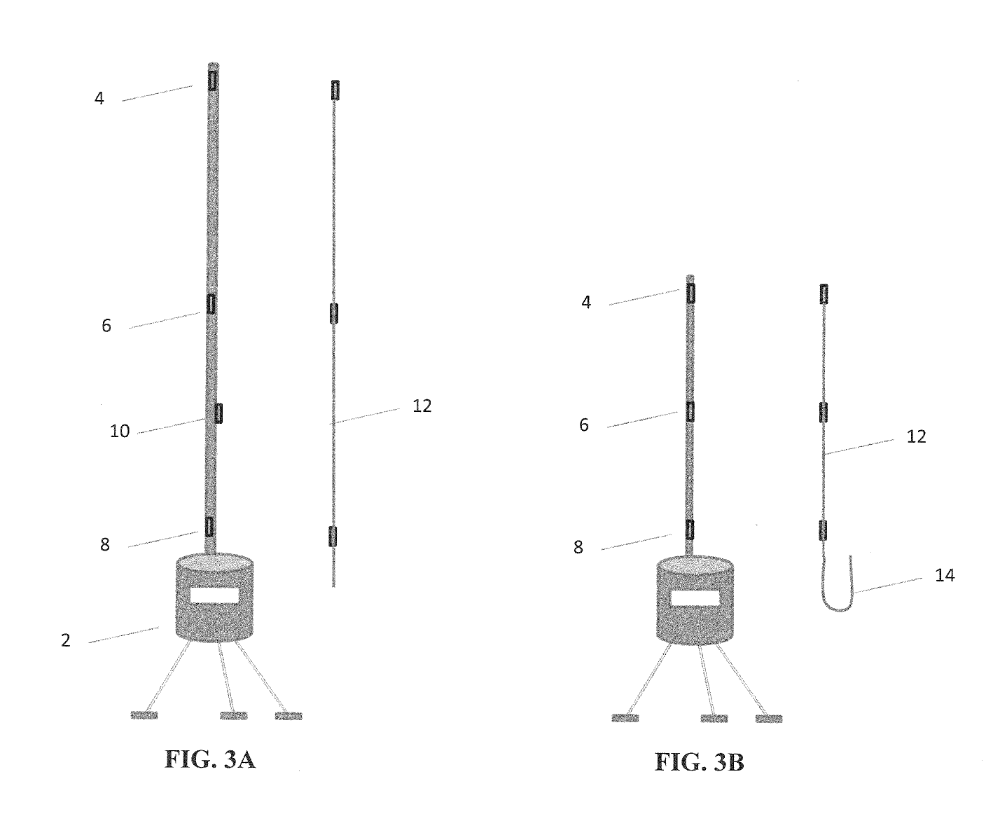

FIG. 3A illustrates one implementation of the invention where a control module 2 is located at a base with an assembly of sensors 4, 6, 8 on a stick 10. The sensors 4, 6, 8 are connected to the control module 2 through a communication wire 12. As illustrated in FIG. 3B, the wire 12 construction may be flexible in nature, so the wire 12 may, for example, appropriately flex 14 when the stick 10 is reduced in size. The stick 10 with the assembly of sensors 4, 6, 8 may be replaced with another sensor stick for the unit to perform measurement for a different environmental sense function.

The control module 2 may be configured for alarm thresholds for each sensor 4, 6, 8. Should the user be interested in the detail of the environmental parameters in a time-stamped fashion, an external memory adapter may allow for storage of time-tagged sensor information. In certain instances, the alarm threshold crossing may warrant immediate attention. As explained in greater detail below, a radio frequency transmitter may exist within the control module or the sensor module depending on the implementation to trigger a contact for remote alarming purposes.

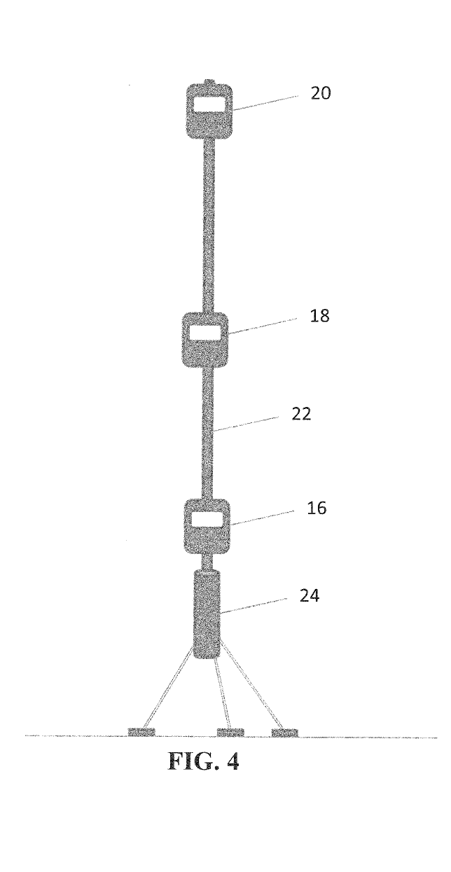

FIG. 4 illustrates an alternative implementation of the invention where the sensor modules 16, 18, and 20 are independent of each other mounted on a flexible stick 22. The base 24 is simply a mechanical support. Each sensor module, explained in greater detail below, may be located at different heights depending on the application. Both the addition of external memory for detailed analysis of time-stamped sensor data and the transmitter for remote alarming purposes mentioned in the previous implementation also exist in each individual sensor module 16, 18, 20 in this implementation.

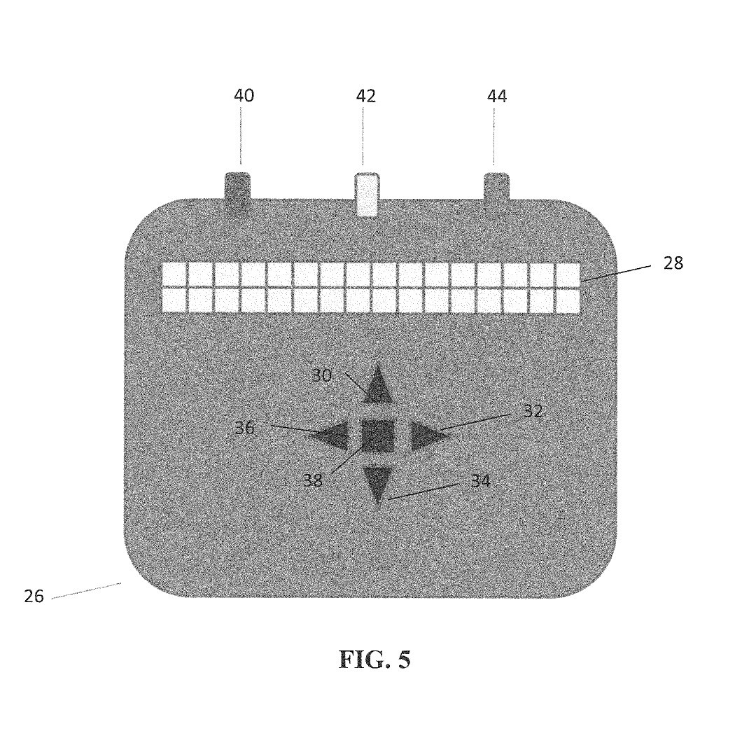

FIGS. 5-8 illustrate a sensor module that is mounted on a stick which allows the user to position the sensor at different places, for example, a computing center, hospital, clean room or general industrial facility. The arrangement allows the user to effectively determine the meteorology of the air at the measurement point. Each figure will now be described in greater detail.

FIG. 5 illustrates a front face view of the sensor module 26 is illustrated which consists of an alphanumeric display 28 and a keyboard 30, 32, 34, 36, 38. This is the principle user interface through which the user programs the sensor module 26. This interface allows for the user to set the set-points for the different environmental parameters. The user is able to choose the set-points from established industry standards or choose custom set-points. For example, there are industry standard limits for temperature and humidity set by the American Society of Heating, Refrigerating and Air-Conditioning Engineers, Inc. (ASHRAE) TC9.9 committee that the Data Center facility managers must adhere to. This committee has recommended certain limits for temperature and humidity that the air going into the servers must comply with. For example, but not limited to, some of these limits may be a part of the “set-points” describe above. In addition, the frequency of the data gathering of the environmental parameters as well as the time period through which this data gathering should be collected is set by the user.

Display indicators 40, 42, 44 are used to indicate the status of the data sampling to the operator. Depending on the threshold that is crossed, the appropriate display indicator is lit or lit with a variable frequency to indicate different conditions to the user. Instead of examining the instrument, the user may simply observe the status of the display indicator to obtain a qualitative input on the state of the environmental status of the environmental parameters. During a walk-through of the facility, indicators like these are more productive than the need to analyze the data collected of the environmental parameters. The user may focus further on the environmental location to diagnose what the potential issues are based on the indicator indication.

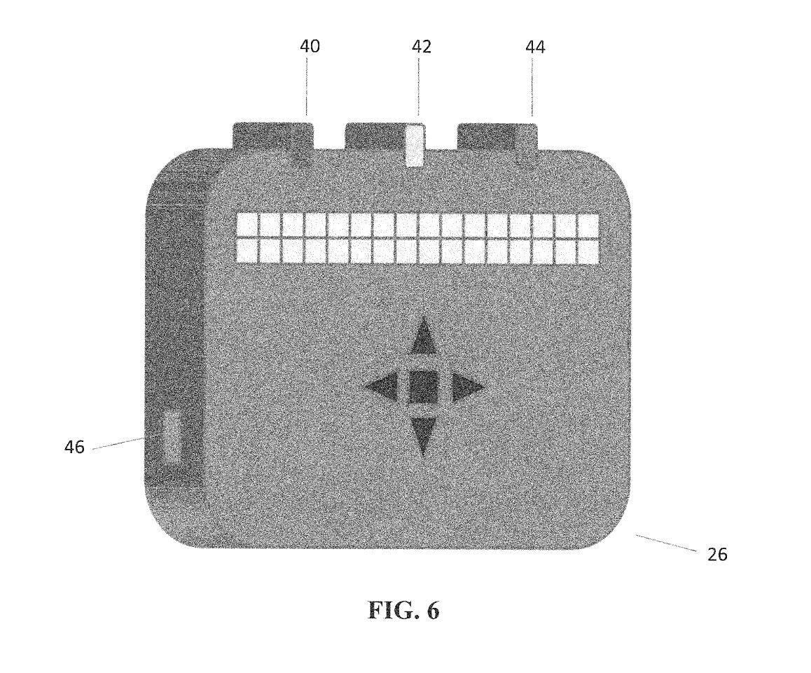

FIG. 6 illustrates a first orientation of the sensor module 26 with an on/off switch 46.

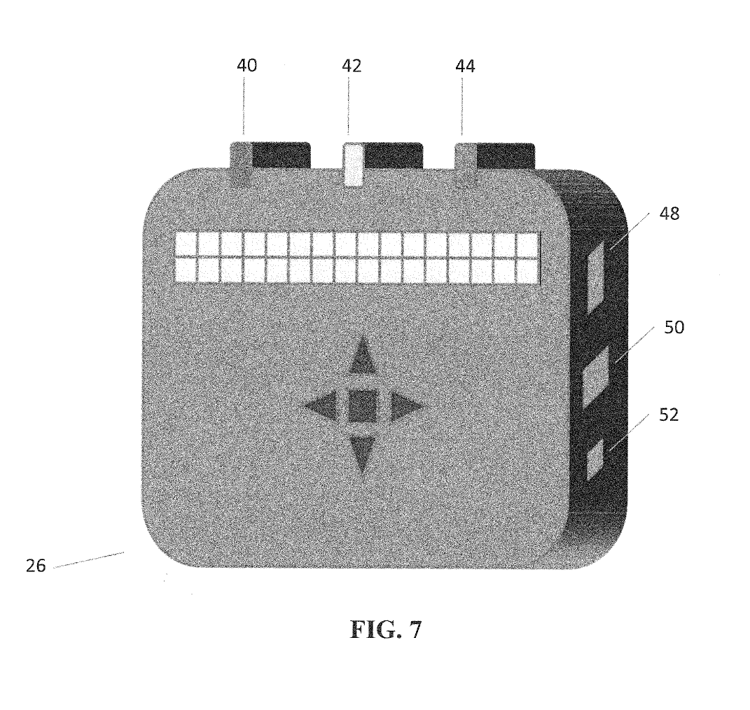

FIG. 7 illustrates examples of various connectors or ports for the sensor module 26. For example, there exists a connector for external memory connection 48. An external memory, such as a flash drive or the like, may be connected to store environmental parameter information or data in the flash drive for post-processing purposes. An Ethernet connector 50 allows the sensor module 26 to communicate the sensor information to web-based processing, such as a cloud. As explained in greater detail below, this is a port through which the data may be transmitted to the cloud. It is noted that one or more aspects of the invention may be implemented in a back-end component, such as a data server or a client computer having a graphical use interface or a web browser through which a user may interact with the sensor module 26 to have a real-time assessment of the data collected in the sensor module. The sensor module 26 thus may be interconnected by any form of medium of digital data communication, such as a local area network (“LAN”), wide area network (“WAN”). A client server relationship may exist between the sensor module 26 and a remote computer through a network. A connector 52 exists for connection of the sensor module 26 to a laptop computer for ease of programming of the sensor module 26. Instead of using the keypads, the user may use the graphical user interface in a laptop computer to configure the sensor module 26.

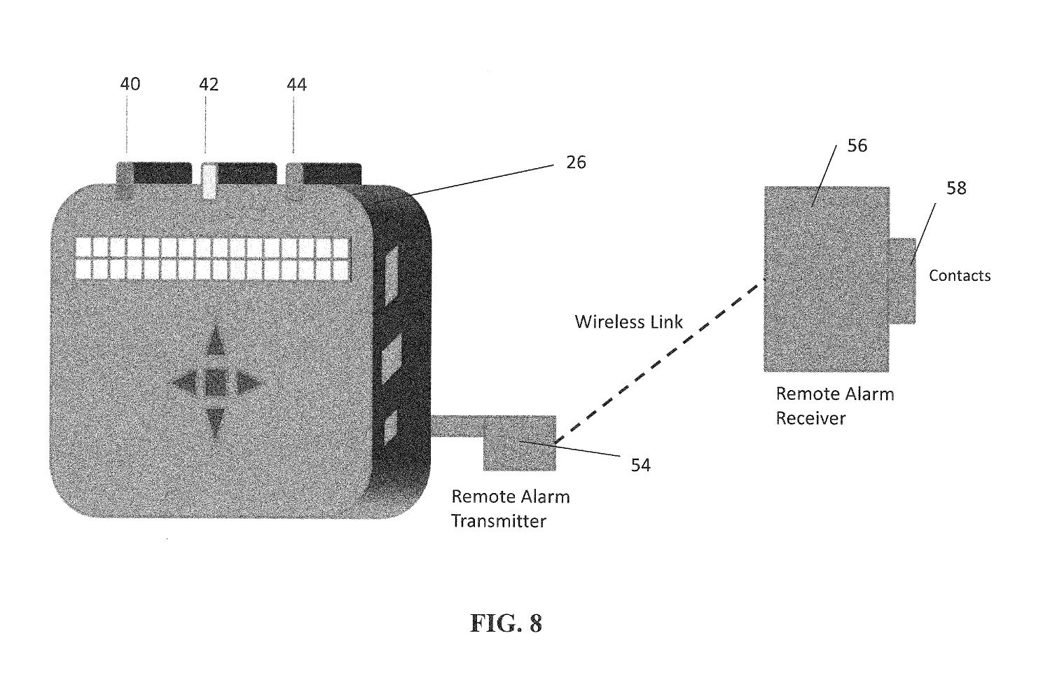

FIG. 8 illustrates the use of a remote alarm transmitter 54 connected to the sensor module 26. For example, the remote alarm transmitter 54 may be plugged into the sensor module 26 and allows the user to send a radio frequency signal to a remote alarm receiver 56. In an alternate implementation the remote alarm transmitter 54 may be incorporated within the sensor or control module itself. The remote alarm receiver 56 may be placed within a distance of 0.5 km from the remote alarm transmitter 54. When the sensor module 26 detects an alarm condition, a radio frequency signal is sent to the remote alarm receiver 56. The remote alarm receiver 56 has a normally open and a normally closed contact 58. Such contacts 58, for example, may be wired to an alarm and/or a building management system for remote warning purposes. The remote alarm receiver 56 indicates a state change to a central master monitoring station by means of contact closure. Detection of such a contact closure would generate an appropriate warning status on a display of the monitoring station for the user to take appropriate action. For example, it will be possible for a user to program one of a plurality of contacts, for example eight such contacts, when the user configures the individual sensor module 26. When an alarm condition is detected by sampling the environment and comparing with the set-point limits, a radio frequency signal is sent to the remote alarm receiver 56 with the code between 0 and 7. Depending on the code, one of eight contacts in the remote alarm receiver 56 will change state. The building management system will be wired to all the contacts. Depending on which contact changed state, the user will be alerted that the specific sensor module 26 detected an alarm or caution condition.

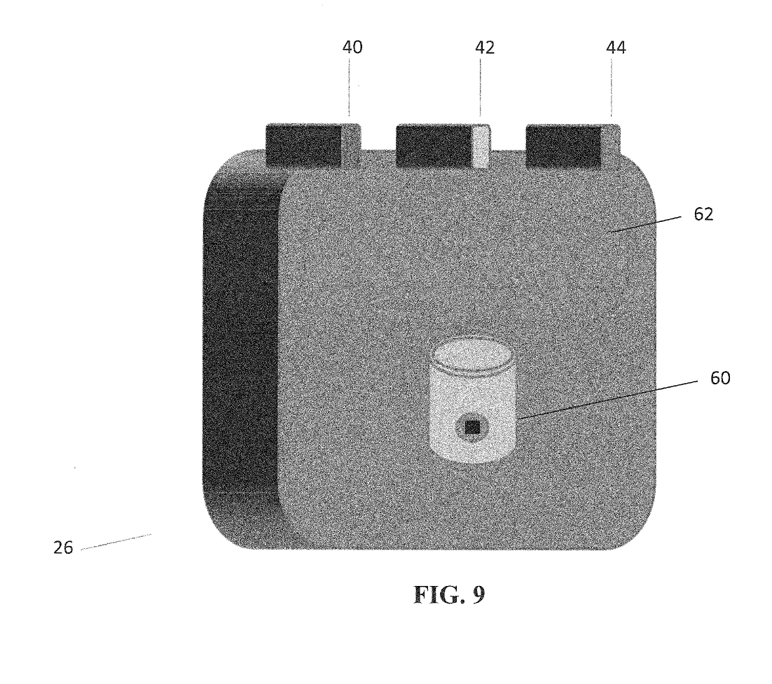

FIG. 9 illustrates a rear view of the sensor module 26. Batteries 60 are replaced through a battery cover 62. The mounting means for the independent sensor module 26 configuration involves an easy mechanical means which may be moved up and down the stick without any issues, and may be fixed at a height desired by the user.

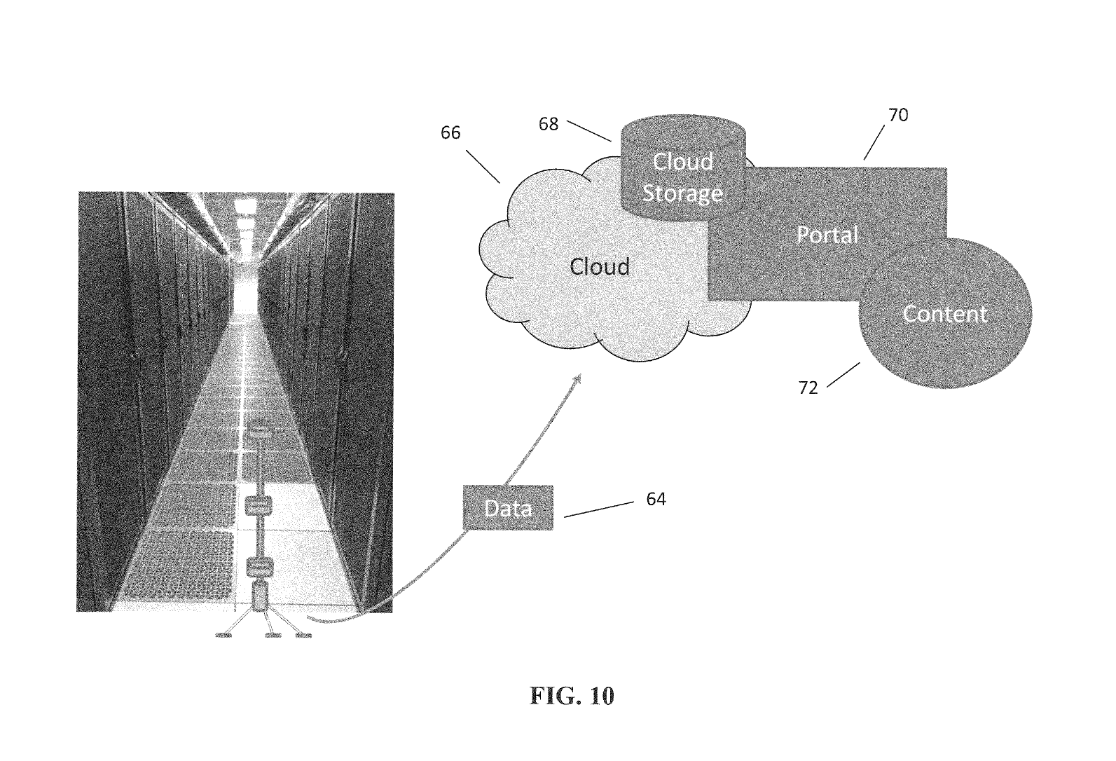

FIG. 10 illustrates the concept of an implementation of the invention using a cloud server 66 having cloud storage 68 and a portal 70 for monitoring the environmental parameter data 64 and information through a remote portal. The content 72 is the intelligence that will parse a lot of data into meaningful information for the user. For example, but not limited to, the data may comprise information of needles, gauges, warning lights, and representing what is going on in the facility. This will vary from site to site and will be user dependent.

While this specification contains many specifics, these should not be construed as limitations on the scope of the disclosure or of what may be claimed, but rather as descriptions of features specific to particular implementations of the disclosure.

Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

A number of implementations have been described. Nevertheless, it will be understood that various modifications may be made without departing from the spirit and scope of the disclosure. Accordingly, other implementations are within the scope of the following claims. For example, the actions recited in the claims may be performed in a different order and still achieve desirable results.

What is claimed is:

1. A portable environmental measurement instrument comprising:
a flexible stick that is adjustable for different heights;
one or more sensor modules coupled to said flexible stick; and
one or more control systems that establish different thresholds for said one or more sensor modules at different heights, said one or more control systems monitors a plurality of environmental parameters in a predetermined sequence and indicates whether at least one environmental parameter is below, within, or above said thresholds of a plurality of set-points through a display indicator.

2. The portable environmental measurement instrument of claim 1, wherein the one or more sensor modules or one or more control systems comprises a transmitter that signals a remote receiver through remote transmittal of said environmental parameter from said one or more sensor modules.

3. The portable environmental measurement instrument of claim 1, wherein said set-points are prescribed limits defined by an industry standard or custom defined by a user.

4. The portable environmental measurement instrument of claim 1, wherein said environmental parameters are stored into a removable memory device.

5. The portable environmental measurement instrument of claim 3, wherein the stored environmental parameters are time-stamped.

6. The portable environmental measurement instrument of claim 1, wherein said environmental parameters comprise combinations of temperature, humidity, noise, air quality, dust, CO₂, CO, radon, ozone, air pressure, air flow, and water.

7. The portable environmental measurement instrument of claim 2, wherein said remote receiver comprises of a pair of contacts connected to a building management system for alarm purposes.

8. The portable environmental measurement instrument of claim 2, wherein said remote receiver is triggered through radio frequency control.

9. The portable environmental measurement instrument of claim 1 further comprising a fan to bring outside air close to said one or more sensor modules to facilitate measurement of said environmental parameters.

10. The portable environmental measurement instrument of claim 1, wherein said one or more sensor modules comprises a user interface, and a plurality of different connection ports.

11. A method of obtaining and communicating environmental parameters, the method comprising:
adjusting a flexible stick for different heights;
coupling one or more sensor modules to said flexible stick; and
establishing different thresholds by one or more control systems for said one or more sensor modules at different heights, said one or more control systems monitors a plurality of environmental parameters in a predetermined sequence and indicates whether at least one environmental parameter is below, within, or above said thresholds of a plurality of set-points through a display indicator.

12. The method of claim 11, wherein the one or more sensor modules or one or more control systems comprises a transmitter that signals a remote receiver through remote transmittal of said environmental parameter from said one or more sensor modules.

13. The method of claim 11, wherein said set-points are prescribed limits defined by an industry standard or custom defined by a user.

14. The method of claim 11, wherein said environmental parameters are stored into a removable memory device.

15. The method of claim 14, wherein the stored environmental parameters are time-stamped.

16. The method of claim 11, wherein said environmental parameters comprise combinations of temperature, humidity, noise, air quality, dust, CO₂, CO, radon, ozone, air pressure, air flow, and water.

17. The method of claim 12, wherein said remote receiver comprises of a pair of contacts connected to a building management system for alarm purposes.

18. The method of claim 12, wherein said remote receiver is triggered through radio frequency control.

19. The method of claim 11 further comprising the step of bringing outside air by a fan to said one or more sensor modules to facilitate measurement of said environmental parameters.

20. The method of claim 11, wherein said one or more sensor modules comprises a user interface, and a plurality of different connection ports.
